(19) United States
(12) Patent Application Publication
(10) Pub. No.: US 2008/0208325 A1
(43) Pub. Date: Aug. 28, 2008

(54) Medical articles for long term implantation

(75) Inventors: Michael N. Helmus, Worcester, MA (US); Barron Tenney, Haverhill, MA (US); Yixin Xu, Newton, MA (US); Kevin M. Alves, New Bedford, MA (US); Frederick H. Strickler, Natick, MA (US); Kathleen M. Miller, Shrewsbury, MA (US); Mark Boden, Harrisville, RI (US)

Correspondence Address: MAYER & WILLIAMS PC, 251 NORTH AVENUE WEST, 2ND FLOOR, WESTFIELD, NJ, 07090, US

(73) Assignee: Boston Scientific Scimed, Inc., Maple Grove, MN (US)

(21) Appl. No.: 12/072,141

(22) Filed: Feb. 25, 2008

Related U.S. Application Data
(60) Provisional application No. 60/903,591, filed on Feb. 27, 2007.

(51) Int. Cl. A61F 2/06

(52) U.S. Cl. 623/1.44; 623/1.13; 623/1.15; 623/1.39

(57) ABSTRACT

According to an aspect of the present invention, long term medical articles are provided which include the following: (a) first and second body contacting (e.g., tissue and/or body-fluid contacting) porous polymeric layers; (b) a polymeric barrier layer disposed between the first and second porous polymer layers; and (c) a reinforcement element. According to another aspect of the present invention, tubular medical articles for long term implantation are provided, which comprise: (a) a reinforcement element; (b) a blood contacting porous polymeric layer having a surface energy ranging between 20 and 30 dynes/cm disposed over an inner surface of the reinforcement element; and (c) an additional porous polymeric layer formed over an outer surface of the reinforcement element.

### STATEMENT OF RELATED APPLICATION

This application claims the benefit of U.S. Provisional Patent Application Ser. No. 60/903,591, filed Feb. 27, 2007, entitled “Medical Articles for Long Term Implantation”, which is incorporated by reference herein.

### FIELD OF THE INVENTION

This invention relates to medical articles for long term implantation, including blood and tissue contacting medical articles for long term implantation.

### BACKGROUND OF THE INVENTION

Various medical articles are known that comprise porous polymeric regions. One particularly beneficial method for forming such medical articles is described in U.S. Pat. No. 4,475,972 to Wong, the disclosure of which is hereby incorporated by reference to the extent that it does not conflict with the present disclosure, in which medical articles are made by a procedure in which fibers are wound on a mandrel and overlying fiber portions are simultaneously bonded with underlying fiber portions. For instance, a polymeric solution can be extruded from a spinneret, forming filaments which are wound onto a rotating mandrel, as the spinneret reciprocates back and forth relative to the mandrel. The drying parameters (e.g., drying environment, solution temperature and concentration, spinneret-to-mandrel distance, etc.) are controlled such that some residual solvent remains in the filaments as they are wrapped upon the mandrel. Upon further evaporation of the solvent, the overlapping fibers on the mandrel become bonded to each other.

Vascular grafts are examples of porous polymeric medical articles, which may be made using the above and other methods. Certain vascular grafts, including small diameter vascular grafts (e.g., grafts with a diameter of 6 mm or less), are known to have very poor long term patency.

Intraluminal stents are commonly inserted or implanted into body lumens, for instance, into a coronary artery after a procedure such as percutaneous transluminal coronary angioplasty (“PCTA”). An example of one such stent 100 is illustrated in FIG. 5. Such stents are used to maintain the patency of the coronary artery by supporting the arterial walls and preventing reclosure or collapse thereof, which can occur after PCTA. Such stents 100 may also have a polymeric coating from which an antiproliferative agent is released to inhibit re-narrowing or restenosis of the blood vessel, which can occur in some patients due to smooth muscle cell proliferation after implantation of the stent 100. Ideally, endothelial cells will grow from the artery wall and over the drug eluting stent struts 100s to form a confluent layer of endothelial cells, particularly after the drug is either completely eluted or the dosage has dropped below an effective antiproliferative level, for example, to prevent smooth muscle proliferation from occurring.

### SUMMARY OF THE INVENTION

According to an aspect of the present invention, medical articles for long term implantation are provided which include the following: (a) first and second body contacting (e.g., tissue and/or body-fluid contacting) porous polymeric layers; (b) a polymeric barrier layer disposed between the first and second porous polymer layers; and (c) a reinforcement element.

An advantage of this aspect of the present invention is that medical articles may be provided, in which the surface pore size and/or porosity can be readily varied, depending on the application at hand.

Another advantage of this aspect of the present invention is that medical articles may be provided which have good biocompatibility, including medical articles that are substantially non-thrombogenic and non-immunogenic.

Another advantage of this aspect of the present invention is that medical articles may be provided in which fluid leakage (e.g., blood, serum, etc.) may be minimized upon puncture of the medical article (e.g., during suturing, AV needle puncture, etc.)

Another advantage of this aspect of the present invention is that medical articles may be provided, in which compliance may be varied from application to application.

Another advantage of this aspect of the present invention is that tubular medical articles may be provided, which have enhanced kink resistance and compression resistance as well as good compliance.

According to another aspect of the present invention, tubular medical articles for long term implantation are provided, which comprise: (a) a reinforcement element; (b) a blood contacting porous polymeric layer having a surface energy ranging between 20 and 30 dynes/cm disposed over an inner surface of the reinforcement element and; and (c) an additional porous polymeric layer formed over an outer surface of the reinforcement element.

An advantage of this aspect of the present invention is that tubular medical articles may be provided, which have good biocompatibility, for example, in the case of tubular vascular articles, encouraging the formation of a covering layer of endothelial cells at their inner surfaces.

These and other embodiments and advantages of the present invention will become immediately apparent to those of ordinary skill in the art upon review of the Detailed Description and Claims to follow.

### BRIEF DESCRIPTION OF THE DRAWINGS

FIG. 1A is a schematic side view of a vascular graft, in accordance with an embodiment of the present invention.

FIG. 1B is a schematic cross-sectional view of the graft of FIG. 1A, taken along line B-B, in accordance with an embodiment of the present invention.

FIG. 2 is a schematic cross-sectional view of the graft of FIG. 1A, taken along line B-B, in accordance with an alternate embodiment of the present invention.

FIGS. 3A and 3B are photographs of vascular grafts, constructed in accordance with the present invention.

FIG. 4A is a schematic cross-sectional view of a vascular stent, in accordance with an embodiment of the present invention.

FIG. 4B is a schematic cross-sectional view of a vascular stent, in accordance with an alternate embodiment of the present invention.

FIG. 5 is a schematic perspective view of a vascular stent in accordance with the prior art.

### DETAILED DESCRIPTION OF THE INVENTION

According to various aspects of the invention, medical articles are provided, which are suitable for long-term implantation and which include at least one polymeric layer.

As used herein, “long-term” implantation means implantation periods of 1 month or more, for example, ranging from 1 month to 3 months to 6 months to 12 months to 24 months or even longer, including the remaining lifetime of the patient. Patients, also referred to as subjects, are vertebrate subjects, more typically mammalian subjects including human subjects, pets and livestock.

As used herein a “layer” (as well as related terms such as “film,” “sheet,” etc.) is a region whose thickness is small compared to its length and width, which need not be planar (e.g., may be a curved, wrinkled, etc.), which may be discontinuous (e.g., patterned), and which may be formed from two or more sub-layers.

As used herein, “porous” polymeric layers for use in the present invention have pore sizes that may vary widely, but whose average diameter is greater than 1 micron (μm) in width, for example, ranging from 1 to 2 to 5 to 10 to 25 to 50 to 100 microns or more. Where pore size is given it is number average pore width and may be measured, for example, using optical microscopy or scanning electron microscopy (SEM). Pores need not be cylindrical. For example, in many embodiments of the invention, porous regions are formed from fibers that overlap at various angles and therefore appear to be randomly distributed and sized upon examination by microscopy. In embodiments where porous polymeric layers are formed by winding fibers onto a rotating substrate, pore size may be defined as average fiber spacing, which may be calculated based on as-deposited fiber width, substrate rotation speed and speed at which the fiber distributor (e.g., a spinneret) traverses the length of the rotating substrate. The porosity of such layers may also vary widely, with 25 to 90% porosity being typical. “Porosity” is defined herein as pore volume (i.e., void space) divided by total volume.

In certain embodiments of the invention, polymeric barrier layers are utilized. As defined herein, a “polymeric barrier layer” is a polymeric layer that is impermeable to cells, although it may be permeable to bodily fluids, which may include, for example, small molecules such as electrolytes and macromolecules such as proteins and polynucleotides. For example, platelets are the smallest blood cells, having typical diameters of about 2 microns. Consequently, to allow water and nutrient permeation while acting as a barrier to cell migration, polymeric barrier layers for use in the present invention have average pores sizes that are 1 micron or less, for example, 1000 nm to 500 nm to 250 nm to 100 nm to 50 nm to 25 nm to 10 nm or less, and are therefore not “porous” as defined hereinabove. Certain polymeric barrier layers for use in the present invention will have no observable pores, even under scanning electron microscopy.

As their names suggest, porous polymeric layers, polymeric barrier layers, polymeric fibers, and so forth, contain polymers, and commonly contain 50 wt % to 75 wt % to 90 wt % to 95 wt % to 97.5 wt % to 99 wt %, or even more polymers.

As defined herein, “polymers” are molecules that contain multiple copies of one or more types of constitutional units, commonly referred to as monomers, and typically contain from 5 to 10 to 25 to 50 to 100 to 500 to 1000 or more of each type of constitutional unit. As used herein, the term “monomers” may refer to the free monomers and to those that have been incorporated into polymers, with the distinction being clear from the context in which the term is used.

Polymers include, for example, homopolymers, which contain multiple copies of a single type of constitutional unit, and copolymers, which contain multiple copies of at least two dissimilar types constitutional units, which units may be present in any of a variety of distributions and include random copolymers, statistical copolymers, gradient copolymers, periodic copolymers (e.g., alternating copolymers), and block copolymers, among others.

Polymers for use in the present invention may have a variety of architectures, including cyclic, linear and branched architectures. Branched architectures include star-shaped architectures (e.g., architectures in which three or more chains emanate from a single molecular region), comb architectures (e.g., architectures having a main chain and a plurality of side chains), dendritic architectures (e.g., arborescent and hyperbranched polymers), and networked (e.g., crosslinked) architectures, among others.

Polymeric layers for use in the present invention may be biostable, biodegradable or a mixture of both, depending on the application, and they are elastomeric in many embodiments.

As defined herein, a “biostable” material is one which remains intact over the period that the medical device is intended to remain implanted within the body. Similarly, as defined herein, a “biodegradable” material is one which does not remain intact over the period which the medical device is intended to remain within the body, for example, due to dissolution, chemical cleavage, etc. of the material.

The “compliance” of a tubular structure, for example, one formed from a single polymeric layer or multiple polymeric layers, is a measure of arterial elasticity and is the percentage of radial change per unit pressure, and is expressed by:

$$C = \frac{Δ D}{D ( Δ P )}$$

where C=compliance, D=diameter of vascular graft at diastole pressure, ΔD=Change in external diameter of vascular graft at systolic and diastolic pressure, ΔP=pulse pressure, usually 120 mm Hg-0 mm Hg for vascular work. See, e.g., “Biologic and Synthetic Vascular Prostheses, Ed. Stanley, J C, Grune & Stratton, 1982, p 191.

The compliance of tubular prosthesis for use in the present invention (including those containing single and multiple polymeric layers) may vary widely, for example, ranging from 0 to 1 to 2 to 3 to 5 to 7 to 10% radial change/mm Hg×0.01. In some embodiments, the compliance approaches a value of 7.4% radial change/mm Hg×0.01, the compliance value for a normal femoral artery, as closely as is possible. Some specific examples of compliance values include about 1.19% radial change/mm Hg×0.01 for expanded polytetrafluoroethylene (e-PTFE) grafts and 1.46% radial change/mm Hg×0.01 for polyethylene terephthalate (PET-DACRON) grafts. See “Biologic and Synthetic Vascular Prostheses”, Ed. Stanley, J C, Grune & Strattton, 1982, p 207). Other examples of compliance values range from 5.95 to 6.10% radial change/mm Hg×0.01 for silastic rubber and polyurethanes, respectively.

In this regard, long term patency of tubular prosthesis, including vascular grafts, is believed to depend on various factors, including prosthesis compliance, as well as other factors such as kink resistance, compression resistance, biocompatibility of the prosthesis including biocompatibility at the blood/prosthesis interface, pore size and porosity at the luminal and abluminal surfaces of the prosthesis, prosthesis permeability, and the degree to which blood leakage occurs at the suture line and at arterio-venous (AV) needle puncture sites, among others.

Polymers for forming polymeric regions in accordance with the present invention may vary widely and include, for example, suitable members selected from the following: polycarboxylic acid polymers and copolymers including polyacrylic acids; acetal polymers and copolymers; acrylate and methacrylate polymers and copolymers (e.g., n-butyl methacrylate); cellulosic polymers and copolymers, including cellulose acetates, cellulose nitrates, cellulose propionates, cellulose acetate butyrates, cellophanes, rayons, rayon triacetates, and cellulose ethers such as carboxymethyl celluloses and hydroxyalkyl celluloses; polyoxymethylene polymers and copolymers; polyimide polymers and copolymers such as polyether block imides and polyether block amides, polyamidimides, polyesterimides, and polyetherimides; polysulfone polymers and copolymers including polyarylsulfones and polyethersulfones; polyamide polymers and copolymers including nylon 6,6, nylon 12, polycaprolactams and polyacrylamides; resins including alkyd resins, phenolic resins, urea resins, melamine resins, epoxy resins, allyl resins and epoxide resins; polycarbonates; polyacrylonitriles; polyvinylpyrrolidones (cross-linked and otherwise); polymers and copolymers of vinyl monomers including polyvinyl alcohols, polyvinyl halides such as polyvinyl chlorides, ethylene-vinyl acetate copolymers (EVA), ethylene-vinyl acrylate copolymers, polyvinylidene chlorides, polyvinyl ethers such as polyvinyl methyl ethers, polystyrenes, styrene-maleic anhydride copolymers, vinyl-aromatic-olefin copolymers, including styrene-butadiene copolymers, styrene-ethylene-butylene copolymers (e.g., a polystyrene-polyethylene/butylene-polystyrene (SEBS) copolymer, available as Kraton® G series polymers), styrene-isoprene copolymers (e.g., polystyrene-polyisoprene-polystyrene), brominated copolymers of isobutylene and paramethylstyrene (e.g., EXXPRO™ from Exxon Mobil), acrylonitrile-styrene copolymers, acrylonitrile-butadiene-styrene copolymers, styrene-butadiene copolymers and styrene-isobutylene copolymers (e.g., polyisobutylene-polystyrene and polystyrene-polyisobutylene-polystyrene block copolymers such as those disclosed in U.S. Pat. No. 6,545,097 to Pinchuk), polyvinyl ketones, polyvinylcarbazoles, and polyvinyl esters such as polyvinyl acetates; polybenzimidazoles; ethylene-methacrylic acid copolymers and ethylene-acrylic acid copolymers, where some of the acid groups can be neutralized with either zinc or sodium ions (commonly known as ionomers); polyalkyl oxide polymers and copolymers including polyethylene oxides (PEO); polyesters including polyethylene terephthalates and aliphatic polyesters such as polymers and copolymers of lactide (which includes lactic acid as well as d-,l- and meso lactide), epsilon-caprolactone, glycolide (including glycolic acid), hydroxybutyrate, hydroxyvalerate, para-dioxanone, trimethylene carbonate (and its alkyl derivatives), 1,4-dioxepan-2-one, 1,5-dioxepan-2-one, and 6,6-dimethyl-1,4-dioxan-2-one (a copolymer of poly(lactic acid) and poly(caprolactone) is one specific example); polyether polymers and copolymers including polyarylethers such as polyphenylene ethers, polyether ketones, polyether ether ketones; polyphenylene sulfides; polyisocyanates; polyolefin polymers and copolymers, including polyalkylenes such as polypropylenes, polyethylenes (low and high density, low and high molecular weight), polybutylenes (such as polybut-1-ene and polyisobutylene), polyolefin elastomers (e.g., santoprene), ethylene propylene diene monomer (EPDM) rubbers, poly-4-methyl-pen-1-enes, ethylene-alpha-olefin copolymers, ethylene-methyl methacrylate copolymers and ethylene-vinyl acetate copolymers; fluorinated polymers and copolymers, including polytetrafluoroethylenes (PTFE), poly(tetrafluoroethylene-co-hexafluoropropene) (FEP), modified ethylene-tetrafluoroethylene copolymers (ETFE), and polyvinylidene fluorides (PVDF), including elastomeric copolymers of vinylidene fluoride and hexafluoropropylene; silicone polymers and copolymers; thermoplastic polyurethanes (TPU); elastomers such as elastomeric polyurethanes and polyurethane copolymers (including block and random copolymers that are polyether based, polyester based, polycarbonate based, aliphatic based, aromatic based and mixtures thereof; examples of commercially available polyurethane copolymers include Bionate®, Carbothane®, Tecoflex®, Tecothane®, Tecophilic®, Tecoplast®, Pellethane®, Chronothane® and Chronoflex®); p-xylylene polymers; polyiminocarbonates; copoly(ether-esters) such as polyethylene oxide-polylactic acid copolymers; polyphosphazines; polyalkylene oxalates; polyoxaamides and polyoxaesters (including those containing amines and/or amido groups); polyorthoesters; biopolymers, such as polypeptides, proteins, polysaccharides and fatty acids (and esters thereof), including fibrin, fibrinogen, collagen, elastin, chitosan, gelatin, starch, glycosaminoglycans such as hyaluronic acid; as well as blends and further copolymers of the above.

Certain beneficial polymeric regions in accordance with the present invention contain block copolymers. Block copolymers are copolymers that contain two or more differing polymer blocks selected, for example, from homopolymer blocks, copolymer blocks (e.g., random, statistical, gradient, and periodic copolymer blocks), and combinations of homopolymer and copolymer blocks. As defined herein, a polymer “block”, refers to a grouping of multiple copies of a single type (i.e., a homopolymer block) or multiple types (i.e., a copolymer block) of constitutional units, commonly 5 to 10 to 20 to 50 to 100 to 200 to 500 to 1000 or more of each type of constitutional unit, and may take on a number of different architectures. As defined herein, a “chain” is an unbranched polymer block.

In certain embodiments, block copolymers for use in the polymeric regions of the present invention contain (a) one or more low T_{g} polymer blocks and (b) one or more high T_{g} polymer blocks. Certain block copolymers having low and high T_{g} polymer blocks are known to possess many interesting physical properties due to the presence of a low T_{g} phase, which is soft and elastomeric at ambient temperature, and a high T_{g} phase, which is hard at ambient temperature. As used herein, “low T_{g} polymer blocks” are those that display a T_{g} that is below ambient temperature, more typically 20° C. to 0° C. to −25° C. to −50° C. or below. Conversely, as used herein, elevated or “high T_{g} polymer blocks” are those that display a glass transition temperature that is above ambient temperature, more typically 50° C. to 75° C. to 100° C. or above. As defined herein, “ambient temperature” is typically 25° C.-45° C., and includes body temperature (e.g., 35° C.-40° C.). T_{g} can be measured by any of a number of techniques including differential scanning calorimetry (DSC), dynamic mechanical analysis (DMA), or dielectric analysis (DEA).

Block copolymer configurations may vary widely and include, for example, the following configurations (in which high T_{g} polymer chains, H, and low T_{g} polymer chains, L, are used for illustrative purposes, although other blocks having different characteristics can clearly be substituted): (a) block copolymers having alternating chains of the type (HL)ₘ, L(HL)ₘ and H(LH)ₘ where m is a positive whole number of 1 or more, (b) multiarm copolymers such as X(LH)ₙ, and X(HL)ₙ, where n is a positive whole number of 2 or more, and X is a hub species (e.g., an initiator molecule residue, a residue of a molecule to which preformed polymer chains are attached, etc.), and (c) comb copolymers having a L chain backbone and multiple H side chains and vice versa (i.e., having an H chain backbone and multiple L side chains).

Some specific examples of low T_{g} blocks include low T_{g} polyalkylene blocks, for example, those comprising ethylene, propylene, butylene, isobutylene, isoprene and/or butadiene monomers, polysiloxane blocks, low T_{g} poly(halogenated alkylene) blocks, low T_{g} polyacrylate blocks, low T_{g} polymethacrylate blocks, low T_{g} poly(vinyl ether) blocks, low T_{g} poly(cyclic ether) blocks, among others.

Some specific examples of high T_{g} blocks include vinyl aromatic blocks, such as those made from monomers of styrene and/or styrene derivatives (e.g., α-methylstyrene, ring-alkylated styrenes, ring-halogenated styrenes or other substituted styrenes where one or more substituents are present on the aromatic ring), high T_{g} polyacrylate blocks, high T_{g} polymethacrylate blocks, poly(vinyl alcohol) blocks, high T_{g} poly(vinyl ester) blocks, high T_{g} poly(vinyl amine) blocks, high T_{g} poly(vinyl halide) blocks and high T_{g} poly(alkyl vinyl ethers), among others.

Specific examples of beneficial block copolymers include those having polyalkylene blocks and poly(vinyl aromatic) blocks, such as block copolymers containing polyisobutylene and polystyrene blocks, for example, polystyrene-polyisobutylene-polystyrene triblock copolymers (SIBS copolymers), described in U.S. Pat. No. 6,545,097 to Pinchuk et al., which is hereby incorporated by reference to the extent that it is not inconsistent with the current disclosure. These copolymers have proven to be valuable elastomers for use implantable or insertable medical device applications due to their excellent strength, biocompatibility and biostability. For example, these copolymers exhibit high tensile strength, which frequently ranges from 13 to 28 MPa or more. Biocompatibility, including vascular compatibility, of these materials has been demonstrated by their tendency to provoke minimal adverse tissue reactions (e.g., as measured by reduced macrophage activity). In addition, these polymers are generally hemocompatible as demonstrated by their ability to minimize thrombotic occlusion of small vessels when applied as a coating on coronary stents. Moreover, stent struts coated with SIBS have been shown to have complete endothelial coverage in <30 days. SIBS copolymers are also biostable, resisting cracking and other forms of degradation throughout the body, including the gastrointestinal tract.

Other specific examples of block copolymers of polyisobutylene and polystyrene include arborescent polyisobutylene-polystyrene block copolymers such as those described in Kwon et al., “Arborescent Polyisobutylene-Polystyrene Block Copolymers-a New Class of Thermoplastic Elastomers,” Polymer Preprints, 2002, 43(1), 266, the disclosure of which is incorporated by reference to the extent that it does not conflict with the present disclosure.

Still other examples of block copolymers having poly(vinyl aromatic) blocks and polyalkylene blocks include polystyrene-poly(ethylene/butylene)-polystyrene (SEBS) block copolymer, available as Kraton™ G series polymers from Kraton Polymers.

It is beneficial in certain embodiments of the invention to select a polymeric region that has a critical surface energy between 20 and 30 dynes/cm. One example of such a material is SIBS, which has a critical surface energy of 25 dynes/cm. Surfaces having a critical surface energy between 20-30 dynes/cm have been shown in work by Dr. Robert Baier and others to provide enhanced biocompatibility, including enhanced thromboresistance. See, e.g., R. E. Baier et al., “Implant Surface Characteristics and Tissue Interaction”, J Oral Implantol, 1988, 13(4), 594-606; Robert Baier et al., “Importance of Implant Surface Preparation for Biomaterials with Different Intrinsic Properties in Tissue Integration in Oral and Maxillofacial Reconstruction”; Current Clinical Practice Series #29, 1986; Robert Baier et al., “Surface Phenomena in In Vivo Environments. Applications of Materials Sciences to the Practice of Implant Orthopedic Surgery”, NATO Advanced Study Institute, Costa Del Sol, Spain, 1984; R. E. Baier et al., “Surface properties determine bioadhesive outcomes: methods and results”, J Biomed Mater Res, 1984, 18(4), 327-355; J. Natiella et al., “Differences in Host Tissue Reactions to Surface-Modified Dental Implants”, 185th ACS National Meeting, American Chemical Society, 1983. In this regard, methods are known for measuring critical surface energy. For example, contact angle methods can be used to produce Zisman plots for calculating critical surface tensions. For further information on measuring critical surface energy, see, e.g., Zisman, W. A., “Relation of the equilibrium contact angle to liquid and solid constitution,” Adv. Chem. Ser. 43, 1964, pp. 1-51; Baier R. E., Shiafrin E. G., Zisman, W. A., “Adhesion: Mechanisms that assist or impede it,” Science, 162: 1360-1368, 1968; Fowkes, F. M., “Contact angle, wettability and adhesion,” Washington D.C., Advances in Chemistry, vol. 43, 1964, p. 1, Souheng Wu, Polymer Interface and Adhesion, Marcel Dekker, 1982, Chapter 5, pp. 169-212. Hence, various polymeric region surfaces may be tested to determine whether or not those surfaces have a critical surface energy within the above criteria.

Numerous techniques are available for forming polymeric regions for use in accordance with the present invention. For example, solvent-based techniques may be used, in which polymeric regions are formed by first providing solutions that contain at least one type of polymer (and any other optional supplemental materials to be processed), and subsequently removing the solvents to form the polymeric regions. The solvents contain one or more solvent species, which are generally selected based on their ability to dissolve the polymer(s) that form the polymeric region, as well as other factors, including drying rate, surface tension, etc. Solvent-based techniques include, but are not limited to, solvent casting techniques, spin coating techniques, web coating techniques, fiber spinning techniques, solvent spraying techniques, dipping techniques, techniques involving coating via mechanical suspension including air suspension, ink jet techniques, electrostatic techniques, and combinations of these processes.

As another example, in embodiments where one or more polymers within the polymeric regions have thermoplastic characteristics, and so long as the polymer(s) (and any other optional supplemental materials to be processed) are sufficiently stable under processing conditions, a variety of thermoplastic processing techniques may be used to form the polymeric regions, including compression molding, injection molding, blow molding, vacuum forming, calendaring, melt spinning, extrusion into sheets, rods, fibers, tubes and other cross-sectional profiles of various lengths, as well as coextrusion into multilayered structures.

In certain embodiments, a solution (where solvent-based processing is employed) or a melt (where thermoplastic processing is employed) is applied to a substrate to form a polymeric region. For example, the substrate may correspond to a region that ultimately forms a part of the medical article, or the substrate may correspond to a template, such as a mold, from which the polymeric region is removed after solidification.

Where employed, fibers may be made by any suitable fiber forming technique, including melt spinning, dry spinning and wet spinning. These processes typically employ extrusion nozzles having one or more orifices, called jets or spinnerets. Fibers having a variety of cross-sectional shapes may be formed, depending upon the shape of the orifice(s) in the spinning die. Some examples of fiber cross-sections include circular, hexagonal, rectangular, triangular, oval, multi-lobed, and annular (hollow) cross-sections. In melt spinning, the polymer compound is heated to melt temperature. In wet and dry spinning the polymer is dissolved in a solvent prior to extrusion. In dry spinning, the extrudate is subjected to conditions whereby the solvent is evaporated, for example, exposure to a vacuum or heated atmosphere (e.g., air) which removes the solvent by evaporation. In wet spinning the jet or spinneret is immersed in a liquid, and as the extrudate emerges, it precipitates from solution and solidifies. Regardless of the technique, the resulting fiber is typically taken up on a rotating substrate, for example, a Teflon®-coated stainless steel mandrel or another suitable take-up device. During take up, the fiber may be stretched (i.e., drawn) to orient the polymer molecules.

In accordance with certain embodiments of the present invention, a dry spinning technique is employed in which a solution containing styrene-isobutylene copolymer is fed (e.g., using a metering pump such as a syringe pump) through one or more fine orifices in a distributor (e.g., those found in a dry spinning die or spinneret). Further details regarding dry spinning of styrene-isobutylene copolymers, may be found in Pub. No. US 2005/0208107, which is hereby incorporated by reference to the extent that there is no conflict between that reference and the present disclosure.

Medical articles in accordance with the present invention that may be formed from fibers are widely varied and include two-dimensional (i.e., open volume) structures (e.g., patches, scaffolding, etc.) and three-dimensional (i.e., closed volume) structures (e.g., tubes, bladders, valves, etc.). They may be formed using any suitable fiber-based construction technique including, for example, a variety of woven and non-woven (e.g., knitted, braided, coiled, randomly wrapped, etc.) techniques. Examples of non-woven techniques include those utilizing thermal fusion, fusion due to removal of residual solvent, mechanical entanglement, chemical binding, adhesive binding, and so forth.

One particularly beneficial method for forming porous tubular three-dimensional structures from fibers is described in U.S. Pat. No. 4,475,972, the disclosure of which is hereby incorporated by reference to the extent that there is no conflict between that reference and the present disclosure, in which such articles are made by a procedure in which fibers are wound upon a mandrel and overlying fiber portions are simultaneously bonded with underlying fiber portions. For instance, a polymer solution may be extruded from distributor (e.g., a spinneret), which may include any number of individual extrusion orifices of various cross-sections (e.g., circular, polygonal, multi-lobed, irregular, etc.). The resulting filaments are wound onto a rotating mandrel, for example, as the distributor reciprocates back and forth relative to the mandrel, or vice versa. Such activity will result in combined rotational and translational movement between the distributor and the mandrel. A suitable number of layers of polymeric fibers are laid down over the mandrel. The drying parameters (e.g., drying environment, solution temperature and concentration, spinneret-to-mandrel distance, etc.) are controlled such that some residual solvent remains in the filaments as they are wrapped upon the mandrel. Upon further evaporation of the solvent, the overlapping fibers on the mandrel become bonded to each other, for example, at various locations where the fibers intersect or otherwise contact each other. Such fiber-to-fiber bonding results when the solvent-containing and only partially set (solidified) fibers engage one another during winding. This engagement may be enhanced, for example, by drawing the extruded fibers (e.g., by selecting appropriate conditions that will uptake the fibers at a speed that is faster than the rate by which they are extruded from the distributor), by formulating the polymer solution to have a high solvent concentration and/or a less volatile solvent, and so forth. This drawing of the extruded fibers also reduces the diameter of the fiber.

The size and shape of pores that are defined by the fibers may be controlled, for instance, by controlling the angle at which the fibers are wrapped upon the mandrel (which depends, for example, on the winding speed of the mandrel relative to the reciprocation speed of the distributor, etc.), by controlling the diameter of the fibers (which depends, for example, on the flow rate of the polymer solution through the spinneret orifice, the draw rate, the solvent content of the polymer solution, etc.), by controlling the degree of flattening of the fibers (e.g., through the use of higher or lower solvent content), and so forth.

The thickness of the layer may be controlled, for instance, by varying the number of wound fiber layers formed on the mandrel, by varying the thickness of the individual fibers, by varying the amount of solvent in the fiber (e.g., if the fiber is wet it may flatten and it may sink into the underlying layer, requiring more fiber passes to reach a desired thickness), and so forth.

In certain embodiments, an electrostatic spinning process is employed. Electrostatic spinning processes have been described, for example, in Annis et al. in “An Elastomeric Vascular Prosthesis”, Trans. Am. Soc. Artif. Intern. Organs, Vol. XXIV, pages 209-214 (1978), U.S. Pat. No. 4,044,404 to Martin et al., U.S. Pat. No. 4,842,505 to Annis et al., U.S. Pat. No. 4,738,740 to Pinchuk et al., and U.S. Pat. No. 4,743,252 to Martin Jr. et al. In these embodiments, electrostatic charge generation components are employed to develop an electrostatic charge between the distributor and the mandrel. For example, the mandrel may be grounded or negatively charged, while the distributor is positively charged. Alternatively, the distributor may be grounded or negatively charged, while the mandrel can be positively charged. The potential that is employed may be constant or variable, and typically ranges from 5 to 11 kV.

As a result of the electrostatic charge that is generated, the polymeric fibers experience a force that accelerates them from the distributor to the mandrel. Also, fibers tend to flap, wobble and/or vibrate. Consequently, structures may be created which have smaller diameter fibers in a more random distribution, relative to the same structures formed in the absence of the electrostatic charge. A lower pressure drop across the spinneret may also be possible during electrostatic spinning. Moreover, contact between the fibers may be enhanced, because the fibers are electrostatically drawn onto the mandrel, in some instances causing the fibers to sink to some extent into underlying fiber layer.

Fibers selected for forming polymeric layers include small diameter fibers, such as those having diameters that are less than 10 microns (μm), for example, ranging from 10 microns to 5 microns to 2.5 microns to 1 micron to 0.5 micron (500 nm) to 0.25 micron (250 nm) to 0.1 micron (100 nm), or less. By using fibers with small diameter, the surface area (per unit weight of the polymeric material forming the fibers) that is exposed to tissue and cells can be increased dramatically.

Within a given polymeric layer, gradients in pore size and porosity may be established by varying parameters such as those described above (e.g., rate of reciprocation between the mandrel and distributor, mandrel rotation rate, flow rate of fiber forming solution, solvent content of the fiber forming solution, etc.).

Moreover, a gradient in polymer composition may be created within a given polymeric layer. In certain embodiments, fibers may be prepared whereby the fiber is composed of differing sections, e.g., varying in polymer compositions along the length of the fiber. For example, (a) where the polymeric layer contains the same polymer or polymers throughout, the molecular weight of the polymer(s) may be varied as the layer is formed, (b) where the polymeric layer contains the same copolymer throughout, the ratio of monomers within the copolymer may be varied as the layer is formed, (c) where the porous polymeric layer contains two or more polymers, the relative amounts of these polymers may be varied as the layer is formed, and so forth. In certain embodiments, two or more polymer types may be layered sequentially with respect to one another, or simultaneously with each other (e.g., with multiple spinnerets being used). In other embodiments, two or more polymer types may be provided within a single fiber, for example, in a core-cladding arrangement, an “islands in the sea” arrangement, or a side be side (e.g., hemispherical) arrangement, among others. See, e.g., U.S. Ser. No. 11/395,964.

Of course porous polymeric layers may be formed using techniques other than fiber spinning. For example, in some embodiments, pores are formed, either in vivo or ex vivo, by introducing a pore generating (poragenic) material.

For example, in some embodiments, a layer is formed from (a) one or more polymers (e.g., homopolymers and/or copolymers) that correspond to the porous layer material, and (b) one or more removable materials (e.g., salts, sugars, dissolvable therapeutic agents, or other dissolvable materials) which leave behind pores upon removal.

For example, in some embodiments, a layer is formed from (a) one or more polymers (e.g., homopolymers and/or copolymers) that correspond to the porous layer material, and (b) one or more removable polymers (e.g., biodegradable polymers, etc.). Mixed polymer compositions are typically phase separated, and the pore size that is ultimately formed in vivo (or ex vivo) will reflect the size of the phase domains that correspond to the one or more removable polymers.

As another example, in some embodiments, a layer is formed using block copolymers that have one or more biodegradable homopolymer or copolymer blocks and one or more biostable homopolymer or copolymer blocks. Examples of such polymers are described in Pub. No. US 2006/0171985 to Richard et al. A specific example of such a polymer for the practice of the present invention is poly(lactic acid)-polystyrene-polyisobutylene-polystyrene-poly(lactic acid) pentablock copolymer, which would yield SIBS upon biodegradation of the poly(lactic acid) blocks. Block copolymer compositions are typically phase separated, and the pore size that is ultimately formed will reflect the size of the phase domains that correspond to the one or more biodegradable homopolymer or copolymer blocks.

As another example, in some embodiments, a layer is formed that contains one or more polymers, one or more solvents, and optionally, one or more therapeutic agents.

Pores can be formed after supercritical CO₂ outgasses from the coating. For example, CO₂ gas may be introduced into the polymer by putting the polymer under pressure. Melt extrusion is an example, whereby CO₂ gas may introduced into the extruder barrel during processing, whereupon the increased temperature and pressure of the extrusion process causes the CO₂ gas to mix with the polymer and become a liquid CO₂ (under sufficient pressure and temperature, the gas reverts to a liquid). Some polymers/gases can be miscible with each other. When the extruded polymer/liquid CO₂ emerges from the end of the extruder die in a defined profile (e.g., a tube, monofilament, etc), the CO₂ liquid sublimes into a gas and disperses into the surrounding atmosphere. As the extrusion pressure on the polymer is released, the CO₂ is released rapidly, leaving behind pores in the polymer where the CO₂ liquid was originally retained. The polymer cools and becomes a solid with pores distributed throughout the profile.

In certain embodiments, two or more porous polymeric layers are provided within the medical articles of the invention, for example, with a polymer layer on one surface configured for contact with a first environment and a polymer layer on an opposing surface configured for contact with a second environment. For instance, one porous polymeric layer may be provided on luminal surface of a tubular prosthesis (e.g., a blood contacting surface of a vascular graft, a vascular stent, or a digestive-fluid contacting surface of a gastrointestinal prosthesis, or a urine contacting surface of a urological prosthesis), and the other porous polymeric layer may be provided on the abluminal surface of the device and thus interface with a surrounding soft tissue environment, for instance, perigraft tissue, an artery, or a body cavity (e.g., the peritoneal cavity). As another example, one porous polymeric layer may be provided on a tissue contacting surface of a two dimensional medical device (e.g., a colonic patch or heart patch), while the other porous polymeric layer may be provided on the opposing surface of the device, thus interfacing with a surrounding soft tissue environment or body cavity (e.g., the peritoneal tissue cavity or pericardium cavity). Note that while many of the specific examples described in the present application are directed to vascular prosthesis or gastrointestinal prosthesis (which includes prosthesis for the mouth, pharynx, esophagus, stomach, pancreas, small intestine, large intestine (colon), rectum and anus), the medical articles of the invention are not so restricted, and will find use throughout the body.

Depending upon the application, the pore size and/or porosity of different polymeric layers within a medical article may (or may not) vary significantly. As a specific example, in a vascular graft exemplified below, a luminal porous polymeric layer is provided with a pore size ranging from 10 to 50 microns, more preferably 20 to 30 microns. (see Goldfarb et al., “Expanded Polytetrafluoroethylene (PTFE): A Superior Biocompatible Material for Vascular Prostheses”, Proceedings of the San Diego Biomedical Symposium, February, 1975, pp 451-456), and an abluminal porous polymeric layer is provided with a pore size ranging from 50 to 100 microns (see R. A. White, “The Effect of Porosity and Biomaterial on the Healing and Long Tern Mechanical Properties of Vascular Prostheses”, Trans. Am. Soc. Art. Internal Organs, 43:95-100, 1988). Pore size and porosity may be varied as discussed above, among other methods.

Similarly, in a vascular stent exemplified below, a luminal porous polymeric layer is provided with a pore size ranging from 10 to 50 microns, more preferably 20 to 30 microns and an abluminal porous polymeric layer is provided with a pore size ranging from 10 to 150 microns, more preferably 60 to 100 microns.

In certain embodiments, the thickness does not exceed 500 microns for any porous layer, to ensure that the ingrown tissue can remain viable by nutrient diffusion.

Moreover, the polymeric content of different polymeric layers within a medical article may (or may not) vary significantly, depending upon the application. For example, the polymer content may vary in the following ways: (a) where the polymeric layers contain the same polymer or polymers, the molecular weight of the polymer(s) may vary between the layers, (b) where the polymeric layers contain the same copolymer, the ratio of monomers within the copolymer may vary between the layers, (c) where the porous polymeric layers contain two or more common polymers, the ratio of these polymers may vary between the layers, (d) one polymeric layer may contain a polymer that is not found in another polymeric layer, for example, the layers may contain completely different polymer(s), one layer may contain one or more polymers in addition to the polymer(s) found in the other layer, and so forth.

As a specific example based on preceding category (b), a vascular graft and a vascular stent are exemplified below in which the luminal and abluminal surfaces each contains a styrene-isobutylene copolymer; however, the luminal surface has a higher proportion of styrene relative to isobutylene than does the abluminal surface.

In particular, as noted above, SIBS triblock copolymers have proven to be valuable elastomers for use in medical devices due, for example, to their excellent strength, biostability and biocompatibility, particularly within the vasculature. Increasing isobutylene content relative to styrene content (or, stated conversely, decreasing styrene content relative to isobutylene content) increases elasticity, but also increases surface tack. On the other hand, decreasing isobutylene content relative to styrene content (or increasing styrene content relative to isobutylene content) reduces surface tack, but increases the stiffness of the material.

In certain embodiments, such as those described below, a surface layer (e.g., the inner layer against the mandrel), may have a decreased isobutylene content so as to reduce surface tack, whereas subsequent layers may have increased isobutylene content to improve compliance which would otherwise be low, due to the high styrene content.

As noted above, the medical articles of the present invention may also contain one or more polymeric barrier layers in certain embodiments.

Polymeric barrier layers may be formed by a variety of processes. For example, polymeric barrier layers may be formed from polymer containing fluids, such as polymeric solutions or polymeric melts, by applying the polymer containing fluid to an underlying template (e.g., a porous polymeric layer such as those described above or a template from which the resulting layer may be removed, such as a mold, mandrel etc.). Polymer containing fluid may be applied, for example, by various application techniques such as those previously described, including, for example, dip coating, spray coating, spin coating, web coating, fiber spinning, and so forth, with multiple applications being possible.

For example, a polymer containing fluid may be applied to a mandrel before or after the formation of a porous polymeric layer such as those described above. Where the polymer containing fluid is applied to the porous polymeric layer, the porous polymeric layer may be removed from the mandrel prior to application of the polymer containing fluid, or it may remain on the mandrel during application (e.g., dip coating, spray coating, etc.).

As a specific example, a polymer solution may be forced through a distributor such as a spray head toward a rotating mandrel. If desired, an electrostatic field may be established between the distributor and the mandrel during application. If the solvent and evaporation conditions are properly selected, the solvent will flash off almost immediately in order to leave a thin polymeric barrier coating on the mandrel or porous layer. In the latter case, such immediate flashing reduces the chance that polymeric solution will wick down into the underlying porous layer and thereby form a coated porous layer, rather than a barrier layer as desired. In other embodiments, the polymer containing solution may be atomized, for example, as described in, U.S. Pat. No. 4,743,252 to Martin, Jr. et al.

As another specific example, polymeric barrier layers may be provided using fiber spinning processes, such as those described above, under conditions where the pores are very small (less than 1 micron) or are not even observable.

In some embodiments, one or more therapeutic agents may be provided within the medical articles of the invention, for example, disposed on or within one or more polymeric layers (e.g., porous polymeric layers, polymeric barrier layers, etc.) within the articles. “Therapeutic agents,” drugs,” “bioactive agents” “pharmaceuticals,” “pharmaceutically active agents”, and other related terms may be used interchangeably herein and include genetic and non-genetic therapeutic agents. Therapeutic agents may be used singly or in combination.

Taking a fibrous porous polymeric region as an example, one or more therapeutic agents may be: (a) provided within the fibers making up the region (e.g., by providing the therapeutic agent(s) within the polymeric solution that forms the fibers or imbibing it into the fibers, either before or after forming polymeric layers from the same), (b) provided within a coating that is sprayed or otherwise provided on the fibers, before forming a porous polymeric region from the fibers, (c) provided within a coating that is sprayed or otherwise provided on the porous polymeric region, after it is formed (e.g., within an adjacent polymeric barrier layer or within a coating that coats the pores of the porous polymeric layer without forming a barrier layer), (d) covalently or non-covalently bound to the surface of the fibers before forming a porous polymeric region from the fibers, (e) covalently or non-covalently bound to the surface of the porous polymeric region after it is formed, (f) covalently or non-covalently bound to the surface of a polymeric barrier layer, and so on.

Taking a poragenic-material-containing layer as an example, compositions may be provided wherein (a) the therapeutic agent is preferentially located in a biodegradable phase of a multiphase composition, whereupon disintegration of the poragenic material assists in controlling the release rate of the therapeutic agent, (b) the therapeutic agent is preferentially located in a biostable phase of a multiphase composition, whereupon disintegration of the poragenic material results in the creation of pores/channels, which assist in controlling the release rate of the therapeutic agent, (c) the therapeutic agent is preferentially located under a polymeric coating, whereupon disintegration of the poragenic material results in the creation of pores/channels, which assist in controlling the release rate of the therapeutic agent, and so forth. As noted above, in some embodiments, the therapeutic agent acts as a poragen (e.g., at certain therapeutic-agent-to-polymer ratios), thereby creating pores/channels as the therapeutic agent elutes from the layer, which pores/channels assist in controlling the release rate of the therapeutic agent.

A wide range of therapeutic agent loadings can be used in conjunction with the devices of the present invention, with the pharmaceutically effective amount being readily determined by those of ordinary skill in the art and ultimately depending, for example, the nature of the therapeutic agent itself, the environment into which the medical article is introduced, that nature of the association between the therapeutic agent and the polymeric region, and so forth.

A wide variety of therapeutic agents can be employed in conjunction with the present invention including those used for the treatment of a variety of diseases and conditions (i.e., the prevention of a disease or condition, the reduction or elimination of symptoms associated with a disease or condition, or the substantial or complete elimination of a disease or condition). Numerous therapeutic agents are described below.

Therapeutic agents may be selected, for example, from the following: adrenergic agents, adrenocortical steroids, adrenocortical suppressants, alcohol deterrents, aldosterone antagonists, amino acids and proteins, ammonia detoxicants, anabolic agents, analeptic agents, analgesic agents, androgenic agents, anesthetic agents, anorectic compounds, anorexic agents, antagonists, anterior pituitary activators and suppressants, anthelmintic agents, anti-adrenergic agents, anti-allergic agents, anti-amebic agents, anti-androgen agents, anti-anemic agents, anti-anginal agents, anti-anxiety agents, anti-arthritic agents, anti-asthmatic agents, anti-atherosclerotic agents, antibacterial agents, anticholelithic agents, anticholelithogenic agents, anticholinergic agents, anticoagulants, anticoccidal agents, anticonvulsants, antidepressants, antidiabetic agents, antidiuretics, antidotes, antidyskinetics agents, anti-emetic agents, anti-epileptic agents, anti-estrogen agents, antifibrinolytic agents, antifungal agents, antiglaucoma agents, antihemophilic agents, antihemophilic Factor, antihemorrhagic agents, antihistaminic agents, antihyperlipidemic agents, antihyperlipoproteinemic agents, antihypertensives, antihypotensives, anti-infective agents, anti-inflammatory agents, antikeratinizing agents, antimicrobial agents, antimigraine agents, antimitotic agents, antimycotic agents, antineoplastic agents, anti-cancer supplementary potentiating agents, antineutropenic agents, antiobsessional agents, antiparasitic agents, antiparkinsonian drugs, antipneumocystic agents, antiproliferative agents, antiprostatic hypertrophy drugs, antiprotozoal agents, antipruritics, antipsoriatic agents, antipsychotics, antirheumatic agents, antischistosomal agents, antiseborrheic agents, antispasmodic agents, antithrombotic agents, antitussive agents, anti-ulcerative agents, anti-urolithic agents, antiviral agents, benign prostatic hyperplasia therapy agents, blood glucose regulators, bone resorption inhibitors, bronchodilators, carbonic anhydrase inhibitors, cardiac depressants, cardioprotectants, cardiotonic agents, cardiovascular agents, choleretic agents, cholinergic agents, cholinergic agonists, cholinesterase deactivators, coccidiostat agents, cognition adjuvants and cognition enhancers, depressants, diagnostic aids, diuretics, dopaminergic agents, ectoparasiticides, emetic agents, enzyme inhibitors, estrogens, fibrinolytic agents, free oxygen radical scavengers, gastrointestinal motility agents, glucocorticoids, gonad-stimulating principles, hemostatic agents, histamine H2 receptor antagonists, hormones, hypocholesterolemic agents, hypoglycemic agents, hypolipidemic agents, hypotensive agents, HMGCoA reductase inhibitors, immunizing agents, immunomodulators, immunoregulators, immune response modifiers, immunostimulants, immunosuppressants, impotence therapy adjuncts, keratolytic agents, LHRH agonists, luteolysin agents, mucolytics, mucosal protective agents, mydriatic agents, nasal decongestants, neuroleptic agents, neuromuscular blocking agents, neuroprotective agents, NMDA antagonists, non-hormonal sterol derivatives, oxytocic agents, plasminogen activators, platelet activating factor antagonists, platelet aggregation inhibitors, post-stroke and post-head trauma treatments, progestins, prostaglandins, prostate growth inhibitors, prothyrotropin agents, psychotropic agents, radioactive agents, repartitioning agents, scabicides, sclerosing agents, sedatives, sedative-hypnotic agents, selective adenosine A1 antagonists, serotonin antagonists, serotonin inhibitors, serotonin receptor antagonists, steroids, stimulants, thyroid hormones, thyroid inhibitors, thyromimetic agents, tranquilizers, unstable angina agents, uricosuric agents, vasoconstrictors, vasodilators, vulnerary agents, wound healing agents, xanthine oxidase inhibitors, and the like.

Numerous additional therapeutic agents useful for the practice of the present invention may be selected from those described in paragraphs [0040] to [0046] of commonly assigned U.S. Patent Application Pub. No. 2003/0236514, the disclosure of which is hereby incorporated by reference to the extent that it does not conflict with the present disclosure.

Some specific beneficial therapeutic agents include antiproliferative agents such as taxanes, including paclitaxel (and particulate forms thereof such as ABRAXANE albumin-bound paclitaxel nanoparticles), rapamycin (sirolimus) and its analogs (e.g., everolimus, tacrolimus, zotarolimus, etc.), Epo D, dexamethasone, angiostatin, estradiol, halofuginone, cilostazole, geldanamycin, ABT-578 (Abbott Laboratories), trapidil, liprostin, Actinomcin D, Resten-NG, Ap-17, abciximab, clopidogrel, Ridogrel, beta-blockers, bARKct inhibitors, phospholamban inhibitors, Serca 2 gene/protein, vascular endothelial growth factors (e.g., VEGF-2), antithrombotic agents (e.g., heparin, hirudin, etc.), resiquimod, imiquimod (as well as other imidazoquinoline immune response modifiers), human apolioproteins (e.g., AI, AII, AIII, AIV, AV, etc.), as well a derivatives of the forgoing, among many others.

Medical articles in accordance with the present invention may also contain one or more reinforcement elements at any of a variety of locations within the same. For example, in the case of tubular medical articles, reinforcement elements may be provided at the inner luminal surface, at the outer abluminal surface, and/or somewhere between the inner and outer surfaces. With respect to medical articles containing two or more porous polymeric layers, for example, the reinforcement structures may be provided at an outer surface of either or both porous polymeric layers, between the porous polymeric layers and so forth.

As used herein, a “reinforcement element” is one that increases the strength (e.g., bend resistance, kink resistance, compression resistance, etc.) of the medical article, commonly by a factor of 2 to 5 to 10 times or more. For example, with tubular medical articles such as vascular prosthesis, such reinforcement elements may be provided to increase kink and/or compression resistance (e.g., to improve short and long term patency).

In various embodiments, such medical articles are strengthened, while at the same time remaining flexible (e.g., to facilitate surgical handling). For this purpose, elastic reinforcement elements may be selected. As defined herein an elastic reinforcement element is one which can elastically recover from tension, compression, torsion and/or bending, with minima plastic/permanent deformation. Typically, elastic reinforcement elements have an elastic modulus (modulus of elasticity) that ranges, for example, from on the order of 1,000 MPa (for a polymer) to 2500 MPa to 5,000 MPa to 10,000 MPa to 25,000 MPa to 50,000 MPa, to 100,000 MPa to 250,000 MPa (for a hard metal).

A variety of materials may be used as reinforcement elements, assuming that they have the proper mechanical characteristics and that they do not adversely affect biocompatibility of the medical article to a significant degree. Materials include non-metallic materials such as homopolymers, copolymers, polymer blends, and polymer composites (e.g., formed using various polymers and bioengineering plastics, such as polypropylene, FEP, PEEK, etc.). Materials also include metallic materials, such as metals (e.g. Ti, Ta), metal alloys comprising iron and chromium (e.g., stainless steels, including platinum-enriched radiopaque stainless steel), alloys comprising nickel and titanium (e.g., Nitinol), alloys comprising cobalt and chromium, including alloys that comprise cobalt, chromium and iron (e.g., elgiloy alloys), alloys comprising nickel, cobalt and chromium (e.g., MP 35N) and alloys comprising cobalt, chromium, tungsten and nickel (e.g., L605), alloys comprising nickel and chromium (e.g., inconel alloys), as well as composite reinforcement elements made using the forgoing. Particularly beneficial in certain embodiments of the invention, are materials having both super elastic and shape-memory characteristics, for example, alloys comprising nickel and titanium (e.g., Nitinol).

In various embodiments, the reinforcement elements include reinforcement elements of solid cross-section, such as solid filaments (which term may be used interchangeably herein with other like terms such as filamentous elements, fibers, wires, and so forth), including filaments which may be formed, for example, using metallic and/or polymeric materials such as those described in the prior paragraph.

In various embodiments, the reinforcement elements can be in the form of ribbons (i.e., flattened filaments), laser cut, plasma etched or chemically etched tubing (e.g., balloon expandable and self-expandable stents, etc.), or laser cut, plasma etched or chemically etched sheets (which may be, for example, rolled into a tube and welded), among others.

The reinforcement elements may be heat treated, as desired, and may posses a shape memory in some embodiments.

Filaments may be employed, for example, as series of hoops, as woven, braided, knitted, coiled, or random filamentous cylinders, and so forth. The mechanical properties of the filamentous reinforcement elements will be affected, for example, by the nature of the material itself and the fiber diameter that is selected, and either or both may be varied to optimize the same. For example, the diameter of these monofilaments may be on the order of 0.025 mm and higher for grafts, depending on the requirements.

In certain embodiments, it may be desirable to vary the degree of reinforcement at different points within the medical article. For example, in the case of tubular prostheses such as vascular grafts, the degree of reinforcement may vary, for instance, as one proceeds circumferentially around the graft or as one proceeds axially along its length. For a filamentous elements, this effect may be achieved by varying filament density (e.g., by varying the winding frequency), filament diameter, or both.

As a specific example, a higher degree of reinforcement may be employed toward the center of the prosthesis to enhance kink resistance and compression resistance; whereas a lower degree of reinforcement may be employed near the ends to enhance ease of suturing. Taking a wound reinforcement element as an example, this may be achieved by increasing the winding frequency near the center of the prosthesis and lowering the winding frequency near the ends.

Where the medical articles in accordance with the present invention are vascular or non-vascular stents or stent grafts, the stent portion of the device may be made from elastic or plastic materials, including polymeric materials and metallic materials, for example, Ti, Ta, stainless steel, PERSS, Nitinol, Elgiloy, MP35N, L605, and other metals, metal alloys and composites, including those described above. For example, the stent portion may be laser cut tubing, laser cut sheets that are then rolled and welded, chemical or plasma etched tubing, chemical or plasma etched sheets that are then rolled and welded, wound wire, wound and welded wire, braided wire, knitted wire, and so forth. A porous polymeric layer may be provided on the outside of the stent, on the inside of the stent, on both sides of the stent, and so forth. The article may also be a partial stent graft, in which the stent portion is located in one section or in several sections, but is not present continuously throughout the graft.

As indicated above, medical articles in accordance with the present invention include closed-volume (hollow) medical articles, such as tubular articles (e.g., vascular and non-vascular grafts, stents and stent grafts, including large and small vascular grafts such as coronary artery bypass grafts, peripheral vascular grafts and endovascular grafts, other tubular structures such as biliary, urethral, ureteral, intestinal and esophageal tubular structures, etc.), as well as various open-volume medical articles such as vascular and non-vascular patches (e.g., patches for hernia repair and patches for the gastrointestinal tract and the urogenital system). Further examples of medical articles include vascular and non-vascular tissue scaffolding, vascular and non-vascular closure devices, for example devices for closure of peripheral and arterio-venous fistula, valve leaflets for heart valves and venous valves, vascular access devices including vascular access ports and arterio-venous access grafts (e.g., devices which are utilized to give frequent arterial and/or venous access such as for antibiotics, total parental nutrition, intravenous fluids, blood transfusion, blood sampling, or arterio-venous access for hemodialysis, and so forth), embolic filters, uterine slings, fabric to join LVADs (left ventricular assist devices) and TAHs (total artificial hearts) to human arteries, and so forth.

In instances where hollow (including tubular) medical articles are provided to reinforce, repair or replace a body lumen (e.g., grafts, stent grafts, patches etc.), their dimensions may be tailored to approximate the dimensions of all or a portion of the body lumen. Examples of body lumens include lumens of the cardiovascular system such as the heart, arteries and veins (e.g., coronary, femoral, aorta, ilial, carotid and vertebro-basilar arteries), lumens of the genitourinary system such as the urethra (including prostatic urethra), bladder, ureters, vagina, uterus, spermatic and fallopian tubes, the nasolacrimal duct, the eustachian tube, lumens of the respiratory tract, such as the trachea, bronchi, nasal passages and sinuses, lumens of the gastrointestinal tract such as the esophagus, gut, duodenum, small intestine, large intestine, colon, biliary and pancreatic duct systems, lumens of the lymphatic system, and so forth.

Hence, hollow medical devices (including any tubular shape, such as those having circular and elliptical cross-sections) for use in the present invention may vary widely in diameter, for example, ranging from 0.5 mm to 1 mm to 2 mm to 5 mm to 10 mm to 20 mm to 50 mm or more in diameter. For instance, tubular articles having diameters ranging from 0.5 to 2 mm may be employed for microvascular work and conduits for nerve regeneration, those having diameters ranging from 2 to 4 mm may be employed for coronary bypass, those having diameters ranging from 2 to 20 mm, may be employed for peripheral vascular grafts, those having diameters ranging from 20 to 50 mm and above may be employed for endovascular and endoluminal vascular grafts, other tubular prosthesis such as esophageal and colonic prosthesis, and so forth. Thus, vascular grafts include grafts that are greater than 6 mm in diameter and those that are less than or equal to 6 mm in diameter.

For tubular structures made using a rotating mandrel, the inside diameter will depend upon the size of the mandrel, with a typical mandrel diameter range being between 0.5 mm to 50 mm or more as noted above. Larger diameter mandrels are also suitable, for example, for forming tubular articles, which may be cut into flattened sheets or otherwise shaped for making two-dimensional (open) structures such as patches and scaffolds.

Where the device is an expandable coronary artery stent, the pre-expansion size of the stent may be, for example, on the order of 1 mm, ranging for example, from 0.5 to 0.75 to 1 to 1.25 to 1.5 mm in diameter. The post-expansion size of the stent, on the other hand, may be on the order of 5 mm, for example, ranging from 3 to 4 to 5 to 6 mm in diameter. The stent may be constructed in its expanded state and then compressed (e.g., onto a balloon) for deployment. Alternatively, the stent may be constructed in its pre-expansion dimensions and expanded in vivo. In the latter case, where a given pore size is desired in vivo, the device may be manufactured with a pore size that is sufficiently small to achieve the desired pore size upon expansion. For example, where a 1 mm coronary stent is constructed, a pore size of 4-6 microns may be formed to achieve a desired pore size of 20 to 30 microns upon deployment to 5 mm. In some embodiments, pores are created that are wider along the axial direction of the stent than along the circumferential direction, with this asymmetry being reduced upon stent expansion. For example, fiber spinning technology is capable of producing pores that are diamond shape in the original configuration, with greater widths in the axial direction than in the circumferential direction, which widths become more equal to one another upon stent deployment. Indeed, one can make diamond shapes pores of practically any dimension by varying the mandrel speed and carriage speed.

Moreover, more complex hollow structures may be formed. For example, by selecting a tapered (i.e., with a gradual diameter change) or stepped (i.e., with an abrupt diameter change) mandrel, a tapered or stepped tubular structure is readily produced. Even more complex structures may be formed using mandrels that may be dissolved, melted, deflated or other otherwise reduced in size for removal after the structure is formed.

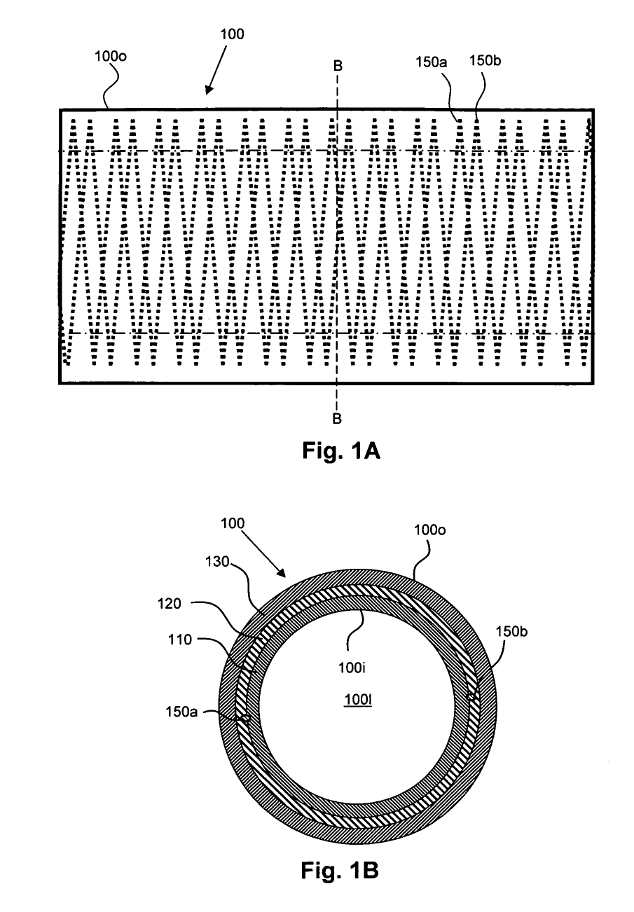

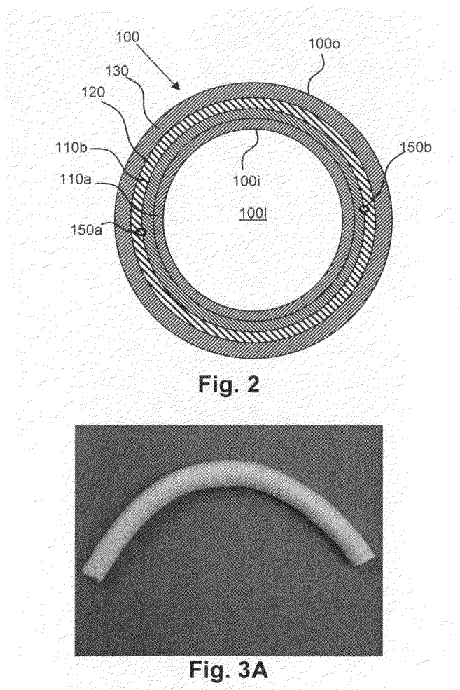

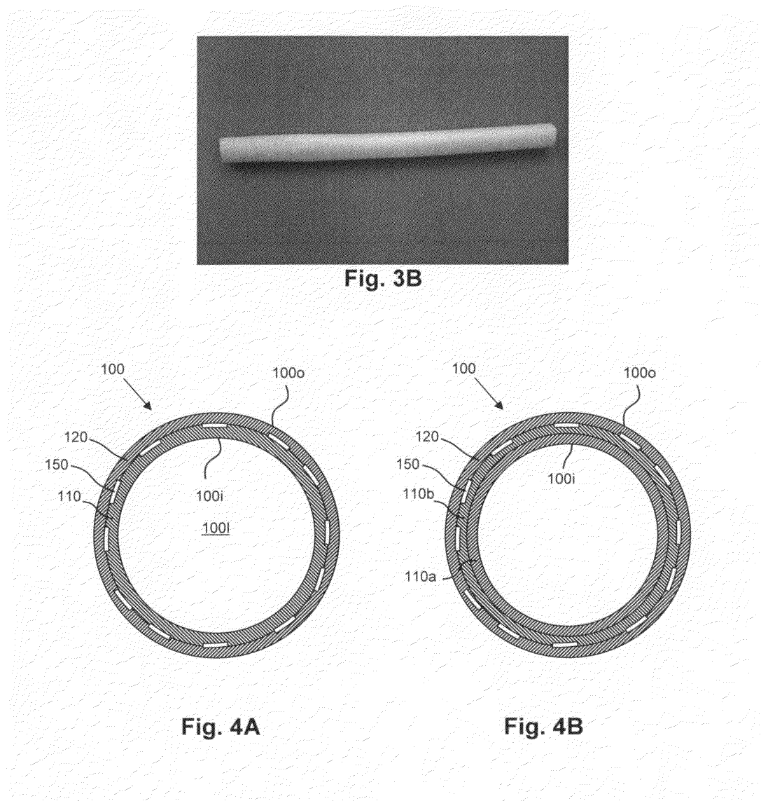

Although it is clear from the above discussion that the present invention is applicable to a wide variety of medical articles, exemplary vascular grafts will now be described in more detail with reference to FIGS. 1A, 1B and 2, and exemplary stents will now be described in more detail with reference to FIGS. 3A and 3B.

FIG. 1A is a schematic side view of a vascular graft 100 in accordance with an embodiment of the present invention. FIG. 1B is a schematic cross-sectional view of the graft 100 of FIG. 1A, taken along line B-B. As discussed in further detail below, this graft incorporates the following features: beneficial pore sizes on its luminal and abluminal (adventitial) surfaces, substantially non-thrombogenic surfaces, an impermeable or semi-permeable polymeric barrier layer within, reduced blood leakage at the suture line and at AV needle puncture sites, enhanced kink and compression resistance, good compliance, as well as the capacity to elute therapeutic agents, if desired.

Turning now to FIGS. 1A and 1B, graft 100 has an outer surface 100o and inner surface 100i, which defines a device lumen 100l. The graft 100 comprises several regions, including an inner (luminal) porous polymeric layer 110, a polymeric barrier layer 120, an outer (abluminal) porous polymeric layer 130, and reinforcement elements 150a, 150b within. Each of these regions will be described in more detail below.

The thickness of the inner porous polymeric layer 110 may vary widely with the thickness depending, on the application. For a small diameter graft, the thickness of the inner layer 110 may be, for example, 100 microns, while that of a larger diameter graft may be, for example, 200 to 300 microns in thickness, giving added strength. For instance, a thicker wall will increase radial hoop strength.

A typical thickness range for vascular grafts is 300 microns to 1000 microns, although other larger or smaller thicknesses may be employed.

The pore size of the inner porous polymeric layer 110 is selected to enhance cell ingrowth and proliferation, thereby encouraging re-endothelialization of the graft and contributing its long-term patency. In the embodiment shown, the inner porous polymeric layer 110 is provided with a pore size ranging from 10 to 50 microns, more typically 20 to 40 microns, as pore sizes within these ranges are known to promote vascular endothelialization. See Goldfarb, et al, “Expanded Polytetrafluoroethylene (PTFE): A Superior Biocompatible Material for Vascular Prostheses”, Proceedings of the San Diego Biomedical Symposium, February, 1975, pp 451-456.

The inner porous polymeric layer 110 is biostable in the embodiment illustrated. If desired, the pores may be filled with a biodegradable or bioresorbable material such as a biodegradable or bioresorbable polymer (several of which are found in the above list of polymers for forming polymeric regions in accordance with the present invention). For example, this may allow cell ingrowth to replace the polymer as it degrades.

In addition to encouraging cell ingrowth and proliferation, the inner porous polymeric layer 110 of the vascular graft 100 should also resist thrombus formation in order to provide a suitable blood interface. Proper polymer selection can greatly assist with reducing thrombus formation. For example, biocompatible polymers such as poly(tetrafluoroethylene), poly(ethylene terephthalate), poly(dimethyl siloxane), SIBS copolymers and poly(lactide-co-glycolide) copolymers (PLGA) are known, which provoke minimal adverse tissue reactions and which minimize thrombotic occlusion.

Thrombus formation may be further suppressed and cell growth may be further enhanced, for example, by releasing or presenting therapeutic agents such as antithrombotic agents and growth factors at the surface 100i that is exposed to blood flow. For instance, these and other agents may be provided between, within, or covalently or non-covalently bound to, (a) the inner porous polymeric layer 110 (e.g., associated with the fiber, admixed with a pore-filling biodegradable material, etc.), (b) the polymeric barrier layer 120, and/or (c) the outer porous polymeric layer 130 (e.g., associated with the fiber, admixed with a pore-filling biodegradable material, etc.).

In certain embodiments, including various embodiments employing SIBS, it may be desirable to employ two sub-layers 110a, 110b as illustrated in FIG. 2, as opposed to a single layer 110 as illustrated in FIG. 1B. The first (luminal) SIBS sub-layer 110a in this particular embodiment may have a pore size of, for example, 10 to 50 microns, more typically 20 to 40 microns. The first (luminal) SIBS sub-layer 110a may have a thickness of, for example, 50 to 150 microns, more typically about 100 microns. Such a layer may be fabricated, for example, from 6 to 10 micron fiber, by employing approximately 200 back and forth cycles at a shuttle speed of 16000 mm/minute and a draw rate (mandrel speed) of 800 rpm for a 6 mm diameter mandrel. Such fiber may be formed, for example, from a 5-45 wt % or more solution of SIBS (30% w/w is presently preferred) in a solvent such as THF. The first SIBS sub-layer 110a may employ a SIBS copolymer having a relatively high styrene content (e.g., 30 to 35 to 40 to 45 to 50 mol %) (with a Shore A Hardness of about 85 to 100 being typical), in order to reduce tack and prevent self-adhesion when the graft 100 is squeezed.

In addition to reduced tack, such high styrene content SIBS has high stiffness. However, this stiffness may compromise the overall compliance of the graft 100, where it is provided in too great a thickness. Ideally, the porous inner region is at least 100 microns thick, but providing high styrene content SIBS in such a thickness would make the material too stiff for certain applications. The second SIBS sub-layer 110b may therefore have a medium styrene content (e.g., 15 to 20 mol %) (with a Shore A Hardness of 25 to 50 being typical), for example, to avoid a loss in the compliance and flexibility of the graft. The second SIBS sub-layer 110b may have a pore size that is similar to that of layer 110a, and may have a thickness that ranges, for example, from 100 to 300 microns, more typically from 150 to 200 microns. A layer of this thickness and pore size may be formed, for example, from a 6 to 10 micron fiber, by employing approximately 300 to 400 back and forth cycles, at a shuttle speed of 16000 mm/minute and a draw rate (mandrel speed) of 800 rpm. Such fiber may be formed, for example, from a 5-45 wt % solution of SIBS (30 wt % presently preferred) in a solvent such as THF.

As an alternative to the discrete jump in properties described in the prior paragraph, an inner porous polymeric layer 110 may be formed in which there is a gradual variation in polymer composition. For example, a porous polymeric layer 110 may be formed in which the fiber-forming solution initially (e.g., at the mandrel) contains SIBS with a high styrene content, but which is gradually replaced with SIBS having a medium styrene content as the thickness of the layer is built up. This layer may have a relatively constant pore size (e.g., 10 to 50 microns) and may have a thickness that ranges, for example, from 150 to 250 microns.

Within the interior of the devices of FIGS. 1A, 1B and 2, one or more reinforcement elements may be provided, for example, on an outside surface of or within the inner porous layer(s) 110, 110a, 110b, on an outside surface of or within the polymeric barrier layer 120, or on an outside surface of or within the outer porous layer 130. The presence of the reinforcing element(s) provides the graft 100 with enhanced strength, including increased kink and compression resistance, thereby improving short and long term patency. For example, such an increase in strength minimizes the opportunity for the lumen walls to touch, thereby reducing thrombus generation.

As previously noted, the reinforcement element(s) may be formed from a variety of materials, for instance, an elastic metal filament such as a Nitinol filament, which may be woven, braided, knitted, coiled or otherwise wrapped around the axis of the structure. For instance, in the specific embodiments illustrated in FIGS. 1A, 1B and 2, two reinforcement members 150a, 150b formed from 0.076 mm superelastic Nitinol filaments are employed, which may be coiled and heat-treated prior to re-coiling them around an underlying layer (e.g., the porous polymeric layer 110 in FIG. 1B or the porous polymeric sub-layer 110b in FIG. 2). Although two coils are illustrated, one, three, four, or more coils may be employed as well as numerous other tubular and non-tubular reinforcement schemes.

As noted above, barrier layers for use in the present invention, such as the barrier layer 120 of the grafts of FIGS. 1A, 1B and 2, are designed to be impermeable to cells, although they may be permeable to the extent that they allow the communication of bodily fluids (which may include, for example, water and small molecules such as electrolytes and macromolecules such as proteins and polynucleotides) between the inner and outer surfaces 100i, 100o of the graft 100. Moreover, the barrier layer 120 also preferably acts as a septum, sealing around sutures during graft implantation and closing AV needle puncture sites. Consequently, the barrier layer 120 typically either has submicron pores or has no observable pores at all. Where submicron pores are present, they may be filled in some embodiments with a biodegradable substance. A variation on this latter concept is to make the entire barrier layer 120 from a biodegradable material (which initially acts as a cellular barrier and septum, as described above, but which is essentially completely absorbed by the body by the time cellular ingrowth from the luminal and adventitial surfaces is complete or nearly complete).

In a specific embodiment, the barrier layer is a SIBS copolymer layer having a low to medium styrene content (e.g., from 5 to 10 to 20 mol % styrene). Such a SIBS copolymer layer is desirable, for example, because it is elastic and therefore contributes to the overall compliance of the device. Moreover, due to its elasticity, the SIBS layer is believed to reduce or eliminate blood leakage at the suture line and at the AV needle puncture sites (e.g., a 16 gauge needle puncture for hemodialysis, etc.), for example, because it is able to recover its deformation and provide a seal around the suture sites (like a septum) and at the puncture sites.

Such a barrier layer 120 may be formed, for example, from a 5-15 wt % solution of SIBS in a solvent such as THF. The solution is applied at room temperature by any appropriate method such as spraying for a sufficient number of passes so as to form a thin barrier layer 120 over the underlying structure, for example, inner porous polymeric layer 110 (FIG. 1B) or sub-layer 110b (FIG. 2), each with surrounding coiled filaments 150a, 150b. Although the solution is applied at room temperature, any temperature below the upper glass transition temperature of the SIBS (about 100° C.) may be used. The drying rate will be affected by increasing or decreasing the temperature.

The grafts 100 of FIGS. 1A, 1B and 2 are further provided with an outer porous polymeric layer 130 which has a pore size ranging, for example, from 60 to 100 microns. This pore size is selected to promote cell in-growth and proliferation at the adventitial surface. By promoting cell in-growth and proliferation, the graft is stabilized within the perigraft tissue, contributing to long term patency and enhancing performance, for example, during AV hemodialysis needle puncture. As with the inner porous polymeric layer 110, in certain embodiments, the pores of the outer porous polymeric layer 130 may be filled with a biodegradable material such as a biodegradable polymer, which is essentially completely absorbed by the body, for example, by the time cellular ingrowth is complete or nearly complete at the adventitial surface.

The thickness of the outer porous polymeric layer 130 may vary widely from application to application, with the thickness depending, for example, on the need for radial hoop strength and soft tissue anchoring, among other factors. A typical range is 50 to 500 microns, although other larger or smaller thicknesses may be employed.

For embodiments in which the porous polymeric layer 130 is formed from SIBS copolymer, the SIBS copolymer selected have an intermediate styrene content (e.g., 15 to 20 mol %) (with a Shore A Hardness 25 to 50 being typical) in order to achieve a less stiff, more flexible graft. The thickness of the porous SIBS layer may range for example, from 100 to 300 microns in thickness. Such a layer may be fabricated, for example, from a 6 to 10 micron fiber, by employing approximately 500 back and forth cycles, at a shuttle speed of 16000 mm/minute and a draw rate (mandrel speed) of 800 rpm. Such fiber may be formed, for example, from a 5-45 wt % solution of SIBS (30% wt % presently preferred) in a solvent such as THF.

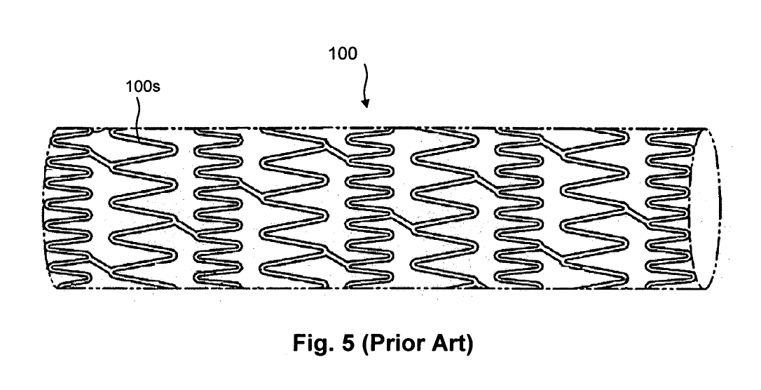

A stent having a design similar to the graft discussed above will now be discussed. FIG. 4A is a schematic cross-sectional view of a coated vascular stent 100 in accordance with an embodiment of the present invention. Coated stent 100 has an outer surface 100o and inner surface 100i, which defines a device lumen 100l. The stent 100 comprises several regions, including (a) an inner (luminal) porous polymeric layer 110, (b) an outer (abluminal) porous polymeric layer 130, and (c) a stent substrate, disposed between the porous polymeric layers 110, 130, which comprises various metallic stent struts 150 (see, e.g., FIG. 5) and acts as a reinforcement element for the device.

The thickness of the inner porous polymeric layer 110 may vary for example, from 1 to 50 microns, although other larger or smaller thicknesses may be employed. In the embodiment shown, the inner porous polymeric layer 110 is provided with a pore size ranging from 10 to 50 microns, more typically 20 to 30 microns, as pore sizes within these ranges are known to promote cellular ingrowth. The inner porous polymeric layer 110 is biostable in the embodiment illustrated. If desired, the pores may be filled with a biodegradable material such as a biodegradable polymer, which is essentially completely absorbed by the body, for example, by the time cellular coverage and ingrowth is complete or nearly complete.

The inner porous polymeric layer 110 of the stent 100 should resist thrombus formation. As noted above, biocompatible polymers such as poly(tetrafluoroethylene), poly(ethylene terephthalate), poly(dimethyl siloxane), SIBS copolymers and PLGA copolymers are known, among others, which provoke minimal adverse tissue reactions and which minimize thrombotic occlusion. Thrombus formation may be further suppressed and cell growth may be further enhanced, for example, by releasing or presenting therapeutic agents such as antithrombotic agents and growth factors at the surface 100i that is exposed to blood flow. For instance, these and other agents may be provided between, within, or covalently or non-covalently bound to the inner porous polymeric layer 110 and/or the outer porous polymeric layer 130.

In certain embodiments, including various embodiments employing SIBS, it may be desirable to employ two sub-layers 110a, 110b as illustrated in FIG. 4B, as opposed to a single layer 110 as illustrated in FIG. 1B. The first (luminal) SIBS sub-layer 110a in this particular embodiment may have a pore size of, for example, 10 to 50 microns, more typically 20 to 40 microns. The first (luminal) SIBS sub-layer 110a may have a thickness of, for example, 50 to 150 microns, more typically about 100 microns. The first SIBS sub-layer 110a may employ a SIBS copolymer having a relatively high styrene content (e.g., 30 to 35 to 40 to 45 to 50 mol %) (with a Shore A Hardness of about 85 to 100 being typical), in order to reduce tackiness. In addition to reduced tack, such high styrene content SIBS has high stiffness. However, this stiffness may compromise the expansion of the stent 100, where it is provided in too great a thickness. Thus a second SIBS sub-layer 110b may have a medium styrene content (e.g., 15 to 20 mol %) (with a Shore A Hardness of 25 to 50 being typical). The second SIBS sub-layer 110b may have a pore size that is similar to that of layer 110a, and may have a thickness that ranges, for example, from 100 to 300 microns, more typically from 150 to 200 microns. As an alternative to the discrete jump in properties, an inner porous polymeric layer 110 may be formed in which there is a gradual variation in polymer composition as discussed above.

As previously noted, the reinforcement element (see, e.g., the stent struts 150) may be formed from a variety of materials, for instance, metallic materials such as stainless steel or Nitinol, among many others. The reinforcement element primarily maintains the patency of the coronary artery by supporting the arterial walls and preventing reclosure or collapse thereof, which can occur after PCTA.

The coated stents 100 of FIGS. 4A and 4B are further provided with an outer porous polymeric layer 130 which has a pore size ranging, for example, from 10 to 50 microns, more typically 20 to 30 microns. This pore size is selected to promote cell in-growth and proliferation from the vessel surface. By promoting cell in-growth and proliferation, the stent is stabilized within the vessel, contributing to long term patency. As with the inner porous polymeric layer 110, in certain embodiments, the pores of the outer porous polymeric layer 130 may be filled with a biodegradable or bioresorbable material such as a biodegradable polymer, which is essentially completely absorbed by the body, for example, by the time cellular ingrowth is complete or nearly complete at the adventitial surface. The thickness of the outer porous polymeric layer 130 may vary widely. A typical range is 50 to 150 microns, although other larger or smaller thicknesses may be employed.

For embodiments in which the porous polymeric layer 130 is formed from SIBS copolymer, the SIBS copolymers selected have a styrene content of up to 50 mol %, for example, 16 to 30 mol % (with a Shore A Hardness of 40 to 92 being typical). The thickness of the porous SIBS layer may range for example, from 50 to 150 microns in thickness.

In some embodiments, a barrier layer like that above (i.e., a layer that is impermeable to cells, although it may be permeable to bodily fluids, which may include, for example, small molecules such as electrolytes and macromolecules such as proteins and polynucleotides) may be provided between the porous abluminal and luminal layers (e.g., adjacent to the reinforcing elements).

### EXAMPLE

SIBS stent grafts like that described in conjunction with FIG. 2 above, i.e., having the following: (a) a first (luminal) sub-layer 110a having a pore size of about 20 to 40 μm and a thickness of about 50 to 100 μm, formed from about 6-10 μm electrostatically spun SIBS fiber having a styrene content of about 30 mol %, (b) a second sub-layer 110b having a pore size of about 20-40 μm and a thickness of about 100 to 150 μm, formed from about 6-10 μm electrostatically spun SIBS fiber having a styrene content of about 17 mol %; (c) a barrier layer 120 formed by spraying an about 10 wt % solution in THF of SIBS having a styrene content of about 17 mol % in an amount sufficient to form a barrier layer 120 over the second sub-layer 110b, and (d) an outer layer 130 having a pore size of about 40-100 μm and a thickness of about 100 to 200 μm, formed from about 6-10 μm electrostatically spun SIBS fiber having a styrene content of about 17 mol %. A heat treated, coiled 0.076 mm superelastic Nitinol filament is provided as reinforcement over the sub-layer 110b in some embodiments (see, e.g., photograph in FIG. 3A), whereas in other embodiments, a coiled filament is not provided (see, e.g., photograph in FIG. 3B). The grafts have an inside diameter of about 6 mm and a length of about 5 to 10 cm or longer.

The above grafts were tested, along with a 6 mm Exxcel Soft ePTFE graft, for water permeability, self sealing on puncture, and suture pull-out using. More particularly, testing was performed in accordance with ISO 7198:1998, Cardiovascular implants—Tubular vascular prostheses. As can be seen from the table below none of the grafts displayed permeability to water. The SIBS graft was found to self-seal more readily than the ePTFE graft upon puncture with a 16 gauge needle. Finally, a greater suture pull-out force was observed for the SIBS grafts, than for the ePTFE graft.

| | Water | | |
|---|---|---|---|
| | Permeability | Self-Sealing on | Suture Pull-out |
| | (cc/min/cm2) | Puncture (cc/min) | (gf/mm) |
| Spun SIBS without | 0 | 25 | 900 |
| Reinforcement | | | |
| Spun SIBS with | 0 | Not Tested | 1950 |
| Reinforcement | | | |
| Exxcel Soft ePTFE | 0 | 192 | Generally about |
| | | | 600-900 |

The unique 3-D spun SIBS structure described above (without coiled filament) was prepared into 6 mm inside diameter×100 mm length tubes for in-vivo testing. After ETO sterilization, spun SIBS tubes were implanted in a modified end-to-end, toe-to-toe, configuration (50 mm lengths) as vascular interpositional grafts in the canine bilateral iliac artery of 2 dogs for one month. Expanded PTFE (6 mm×50 mm length) was used as the bilateral control. An additional six SIBS tubes were similarly implanted as vascular grafts in the canine bilateral iliac artery in 6 dogs expected to survive for six months, again using Expanded PTFE as the bilateral control.

During cutting, the SIBS graft was found to somewhat stick to itself, but this is not at issue with the filament reinforced graft.

During implantation, the surgeon found that the sutures created relatively large holes in the ePTFE graft around the suture penetration area, but not in the SIBS graft, which exhibited a self-sealing quality. He also found that the SIBS graft sutured more readily than the ePTFE. For example, the suture was found to have some drag when moved across the SIBS graft material an indication of the SIBS self-sealing qualities. Moreover, because the SIBS graft is flexible, it is more conformable to the host artery at the anastamoses and allows the surgeon to sew at difficult angles, whereas the ePTFE required that the surgeon sew at more restricted angles. Due to the SIBS' flexibility, the surgeon was able to direct the orientation of the SIBS graft to make suturing easier. The surgeon found the feel of the SIBS graft to be much like that of a blood vessel.

A minute amount of through-the-wall bleed-through was observed at the SIBS graft, when the clamps were released, but there was no accrual of blood around the graft.

At the end of the 1 month period, 2 canines were sacrificed. Patency was 50% (½) for the SIBS grafts and 50% (½) for the ePTFE controls. As both the control and the SIBS graft both occluded at the same time, the root cause was found to be a procedural error and not related to the SIBS or ePTFE grafts. At the end of the 6 month period, the remaining 6 canines were sacrificed. Patency was 83% (⅚) for the SIBS grafts and 100% ( 6/6) for the ePTFE controls. Hence, overall patency for both the 1 and 6 month grafts was 75% ( 6/8) for the SIBS grafts and 83% (⅞) for the ePTFE grafts. The patency for the SIBS test graft was essentially equivalent to the patency for the ePTFE grafts, and the patency for the ePTFE grafts was typical for this material.

Moreover, at six months, the endothelial cell coverage for the SIBS grafts was approximately 93%, whereas the endothelial cell coverage for the ePTFE grafts was 55%.

Although various embodiments are specifically illustrated and described herein, it will be appreciated that modifications and variations of the present invention are covered by the above teachings and are within the purview of the appended claims without departing from the spirit and intended scope of the invention.

What is claimed is:

1. A medical article comprising:
(a) first and second body contacting filamentous porous polymeric layers;
(b) a polymeric barrier layer disposed between the first and second porous polymer layers; and
(c) a reinforcement element, wherein said medical article is adapted for long term implantation.

2. The medical article of claim 1, wherein said medical article is a blood contacting medical article.

3. The medical article of claim 1, wherein said medical article is in the form of a sheet.

4. The medical article of claim 1, wherein said hollow medical article is a tubular medical article.

5. The medical article of claim 1, wherein said first and second porous polymeric layers are formed from polymeric fibers.

6. The medical article of claim 1, wherein said first and second porous polymeric layers are biostable polymeric layers.

7. The medical article of claim 6, wherein pores of at least one of said first and second porous polymeric layers are filled with a biodegradable or bioresorbable material.

8. The medical article of claim 1, wherein said polymeric barrier layer is a biostable barrier layer.

9. The medical article of claim 1, wherein said first porous polymeric layer, said second porous polymeric layer, and said polymeric barrier layer each comprises a copolymer comprising poly(styrene) blocks and polyisobutylene blocks.

10. The medical article of claim 9, wherein said first porous polymeric layer changes in composition over its thickness, and wherein the styrene content of the first porous layer decreases as one proceeds radially into the layer starting from the luminal surface of the device.

11. The medical article of claim 1, wherein said reinforcement element comprises (a) a cut or etched tube or (b) a cut or etched sheet which is optionally rolled into the shape of a tube.

12. The medical article of claim 1, wherein said medical article is a tubular medial article and said reinforcement element is a coiled filament.

13. The medical article of claim 12, wherein the reinforcement element is a coiled elastic filament such that said medical article may be elastically elongated by at least 100% in a longitudinal dimension ex vivo.

14. The medical article of claim 13, wherein the elastic filament has an elastic modulus that ranges from 1,000 MPa to 250,000 MPa.

15. The medical article of claim 13, wherein the elastic filament is selected from composite and non-composite filaments comprising one or more of the following materials: titanium metal, tantalum metal, alloys comprising iron and chromium, alloys comprising nickel and titanium, alloys comprising cobalt and chromium, alloys comprising cobalt, chromium and iron, alloys comprising nickel, cobalt and chromium, alloys comprising cobalt, chromium, tungsten and nickel, and alloys comprising nickel and chromium.

16. The medical article of claim 1, wherein said filamentous reinforcement element is disposed between the first and second porous polymer layers.

17. The medical article of claim 1, wherein said medical article is a vascular graft, wherein said first polymeric layer is positioned at a luminal surface of said medical article, and wherein said second polymeric layer is positioned at an abluminal surface of said medical article.

18. The medical article of claim 17, wherein said first layer has a pore size ranging between 10 and 50 microns and said second polymeric layer has a pore size ranging between 50 and 100 microns.

19. The medical article of claim 17, wherein said first porous polymeric layer, said second porous polymeric layer, and said polymeric barrier layer each comprises copolymer comprising poly(styrene) blocks and polyisobutylene blocks, and wherein said copolymer in said first porous polymeric layer comprises 15 to 50 mole % styrene, said copolymer in said second polymeric layer comprises 15 to 50 mole % styrene, and said copolymer in said polymeric barrier layer comprises from 5 to 50 mole % styrene.

20. The medical article of claim 17, wherein said first porous polymeric layer comprises a first luminal porous polymeric sub-layer and a second porous polymeric sub-layer, wherein said first porous polymeric sub-layer, said second porous polymeric sub-layer, said polymeric barrier layer, and said second porous polymeric layer each comprises a copolymer that comprises poly(styrene) blocks and polyisobutylene blocks, and wherein said copolymer in said first polymeric sub-layer comprises 30 to 50 mol % styrene, said copolymer in said second porous polymeric sub-layer comprises 15 to 20 mol % styrene, said copolymer in said second polymeric layer comprises 15 to 20 mol % styrene, and said copolymer in said polymeric barrier layer comprises from 5 to 15 mol % styrene.

21. The medical article of claim 20, wherein said first and second polymeric sub-layers each has a pore size ranging from 20 to 30 microns, and wherein said second polymeric layer has a pore size ranging from 60 to 100 microns.

22. The medical article of claim 1, wherein said medical article is a stent graft, wherein said first polymeric layer is positioned at a luminal surface of said medical article, wherein said second polymeric layer is positioned at an abluminal surface of said medical article, and wherein said reinforcement element comprises a stent.

23. A medical article comprising:
(a) a reinforcement element;
(b) a blood contacting porous polymeric layer disposed over an inner surface of said reinforcement element, said blood contacting porous polymeric layer having a surface energy ranging between 20 and 30 dynes/cm; and
(c) an additional porous polymeric layer formed over an outer surface of said reinforcement element, wherein said medical article is adapted for long term implantation.

24. The medical article of claim 23, wherein said reinforcement element comprises a vascular stent.

25. The medical article of claim 23, wherein said medical article is a vascular graft and wherein said blood contacting porous polymeric layer has a pore size between 10 and 50 μm.

26. The medical article of claim 23, wherein said medical article is an expandable stent and wherein the blood contacting porous polymeric layer has a pore size between 10 and 50 μm after expansion.

27. The medical article of claim 23, wherein said blood contacting porous polymeric layer is formed from polymeric fibers.

28. The medical article of claim 23, wherein said medical article further comprises an antiproliferative agent.

29. The medical article of claim 28, wherein the antiproliferative therapeutic agent is selected from taxol, sirolimus and sirolimus analogs.

30. The medical article of claim 23, wherein said blood contacting porous polymeric layer and said additional porous polymeric layer each comprises copolymer comprising polystyrene blocks and polyisobutylene blocks.
